SEQUENCE LISTING (2 sequences)

SEQ ID NO: 1 - protein, 9 aa, artificial sequence
  synthetic peptide
LAGLIDADG

SEQ ID NO: 2 - protein, 6 aa, artificial sequence
  synthetic peptide
GIYYIG

(19) United States
(12) Patent Application Publication
(10) Pub. No.: US 2005/0123944 A1
(43) Pub. Date: Jun. 9, 2005

(54) Methods and compositions related to the use of sequence-specific endonucleases for analyzing nucleic acids under non-cleaving conditions

(75) Inventors: Lori Neely, Woburn, MA (US); Maria Hackett, Newburyport, MA (US)

Correspondence Address: Maria A. Trevisan;Wolf, Greenfield & Sacks, P.C., 600 Atlantic Avenue, Boston, MA, 02210, US

(73) Assignee: U.S. Genomics, Inc., Woburn, MA (US)

(21) Appl. No.: 10/898,712

(22) Filed: Jul. 23, 2004

Related U.S. Application Data
(60) Provisional application No. 60/492,143, filed on Aug. 1, 2003.

(51) Int. Cl. C12Q 1/68; C12N 9/22; C07K 16/46

(52) U.S. Cl. 435/6; 435/199; 530/391.1

(57) ABSTRACT

The invention relates to methods, products and systems for analyzing nucleic acid molecules using a nucleic acid binding protein such as a sequence-specific endonuclease. The methods can be used to obtain sequence information about the nucleic acid molecules.

### RELATED APPLICATIONS

This application claims priority under 35 U.S.C. §119 to U.S. Provisional Patent Application Ser. No. 60/492,143, filed Aug. 1, 2003, the entire contents of which are hereby incorporated by reference.

### FIELD OF THE INVENTION

The invention relates to analysis of nucleic acid molecules using endonucleases under conditions in which sequence-specific binding but not cleavage is favored.

### BACKGROUND OF THE INVENTION

Restriction endonucleases are bacterial enzymes that serve to protect the bacterial cell from invasion by foreign DNAs. These enzymes recognize specific sequences within a DNA molecule, referred to as recognition sites or sequences, and catalyze the cleavage of the phosphodiester backbone within or near these sites. Restriction modification (RM) systems include a restriction endonuclease and a cognate DNA methyltransferase (Mtase). The Mtase catalyzes the covalent attachment of a methyl group to a specific nucleotide within the restriction enzyme recognition site. This methylation protects the host DNA from restriction, while foreign unprotected DNAs will be cleaved. There are at least three types of RM systems, referred to as type I, II and III. Type II restriction enzymes have proven an invaluable tool for the sequence-specific excisions required in recombinant DNA technologies. Because of their utility, they have been the subject of an exhaustive amount of research. Over 3000 type II restriction endonucleases have been identified.

### SUMMARY OF THE INVENTION

The invention is premised in part on the finding that proteins capable of binding to nucleic acid molecules without modifying such molecules can be exploited in labeling and sequencing strategies. Various nucleic acid binding proteins can be used for this purpose provided they possess sequence-specific binding capacity. These nucleic acid binding proteins may be DNA or RNA binding proteins including but not limited to polymerases (e.g., DNA polymerase or RNA polymerase), methylases, transcription factors, and in a particularly important embodiment restriction endonucleases.

Thus, in one aspect, the invention provides a method for analyzing a nucleic acid molecule comprising contacting a nucleic acid molecule with a detectable nucleic acid binding protein in a binding condition for a time sufficient for the nucleic acid binding protein to bind to the nucleic acid molecule in a sequence-specific manner, and detecting the bound detectable nucleic acid binding protein. The nucleic acid binding protein may be inherently detectable or it may be extrinsically manipulated in order to be detectable, as described in greater detail below. In important embodiments, the nucleic acid binding protein is a DNA binding protein. The nucleic acid binding protein may be selected from the group consisting of a polymerase, a methylase, a transcription factor, mismatch repair enzymes, chromatin modifying complexes, and proteins involved in RNA interference.

The contacting occurs under binding conditions that allow the nucleic acid binding protein to bind to the nucleic acid molecule but preclude other enzymatic activity of the nucleic acid binding protein. Thus, in one embodiment, the nucleic acid binding protein is a methylase and the binding condition comprises a methylase inhibitor. The methylase inhibitor may be a DNA methylase inhibitor such as 5-azacytidine (5-aza), 5-aza-2′deoxycytidine (also known as Decitabine in Europe), 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, 5-fluoro-2′deoxycytosine, sinefungin, and homocysteine.

In another embodiment, the nucleic acid binding protein is a polymerase and the binding condition lacks a primer or a population of unincorporated nucleotides, thereby precluding its ability to synthesize and thus translocate along the nucleic acid molecule.

The invention is further premised on the observation that under certain conditions, sequence-specific restriction endonucleases are able to bind to a nucleic acid molecule in a sequence-specific manner but are not able to cleave the nucleic acid molecule. Restriction endonucleases, for example, usually require a sufficient concentration of magnesium divalent cations in order to cleave a nucleic acid molecule. It has now been discovered according to the invention that restriction endonucleases will bind to a nucleic acid molecule in a sequence-specific manner but will not cut the nucleic acid molecule in the absence of magnesium divalent cations, even though they may in the presence of calcium divalent cations. These enzymes similarly will not cleave a nucleic acid molecule in an insufficient concentration of Mg²⁺. Thus, Mg²⁺ concentrations that are insufficient for nucleic acid cleavage can also be used.

Thus, in another aspect, the invention provides a method for analyzing a nucleic acid molecule, comprising contacting a nucleic acid molecule with a detectable sequence-specific endonuclease in a non-cleaving condition for a time sufficient for the sequence-specific endonuclease to bind to the nucleic acid molecule in a sequence-specific manner, and detecting the bound detectable sequence-specific endonuclease. The sequence-specific endonuclease may be inherently or intrinsically detectable or it may be extrinsically manipulated in order to be detectable.

The sequence-specific endonuclease may be a restriction endonuclease, such as a type II restriction endonuclease. Examples of type II restriction endonuclease include but are not limited to BamHI, BglI, BglII, EcoRI, EcoRV, MunI, PvuII, HaeIII, HinPI, NotI, PmeI and EagI.

In one embodiment, the non-cleaving condition comprises a Mg²⁺ concentration that does not allow cleavage of the nucleic acid molecule. In another embodiment, the non-cleaving condition lacks Mg²⁺ (i.e., a zero concentration of Mg²+). The non-cleaving condition preferably comprises the presence of a divalent cation selected from the group consisting of Ca²⁺, Co²⁺ and Mn²⁺. In important embodiments, the non-cleaving condition comprises the presence of Ca²⁺.

It has been found that the relative concentrations of Mg²⁺ and Ca2+ also control the extent of binding and cleaving of the nucleic acid molecule by an endonuclease. Accordingly, the non-cleaving condition may comprise a Ca2+ concentration that exceeds a Mg²⁺ concentration. In some embodiments, the Ca²⁺ concentration exceeds the Mg²⁺ concentration by at least 50-fold, at least 100-fold, at least 500-fold, at least 1000-fold, at least 2000-fold and at least 5000-fold. In still other embodiments, the Ca²⁺ concentration is about 5 nM. Preferably, the Mg²⁺ concentration is insufficient to cleave the nucleic acid molecule in the presence or absence of Ca²⁺.

Several embodiments relate equally to the various aspects of the invention and these are described below.

The nucleic acid molecule may be a DNA such as a genomic DNA (e.g., nuclear DNA or mitochondrial DNA) or cDNA, or an RNA such as mRNA, rRNA, snRNA, RNAi, miRNA or siRNA. In important embodiments, the nucleic acid molecule is a non in vitro amplified nucleic acid molecule. The nucleic acid may be non-linearized prior to analysis in the various methods of the invention. Alternatively, it may be linearized prior to or during the analysis. In some instances the method is based on determining the presence or absence of a bound sequence-specific endonuclease or nucleic acid binding protein, while in others the method is based on determining the location of one of more bound sequence-specific endonucleases or nucleic acid binding proteins.

In still other embodiments, the nucleic acid binding protein or sequence-specific endonuclease is labeled with a detectable label, and this labeling can be covalent or non-covalent (e.g., ionic). In some important embodiments, the nucleic acid binding protein or sequence-specific endonuclease is labeled with a single detectable label. The detectable label in some instances is also preferably a non-bead label or a non-fluorescently labeled bead.

The detectable label may include but is not limited to a fluorescent molecule, a chemiluminescent molecule, a radioisotope, an enzyme substrate, a biotin molecule, an avidin molecule, an electrical charged transducing molecule, a nuclear magnetic resonance molecule, a semiconductor nanocrystal, an electromagnetic molecule, an electrically conducting particle, a ligand, a microbead, a chromogenic substrate, an affinity molecule, a quantum dot, a protein, a peptide, a nucleic acid, a carbohydrate, an antibody, an antibody fragment, an antigen, a hapten, and a lipid.

The bound nucleic acid binding molecule or sequence-specific endonuclease may be detected using a detection system including but not limited to a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system. The detectable label may also be defined as one that is detected using any of the afore-mentioned detection systems.

The nucleic acid molecule may be additionally labeled with a detectable label, such as but not limited to a backbone label or an end label. Alternatively, it may be labeled in order to identify “landmarks” such as centromeres, repetitive sequences, and the like.

Preferably the bound nucleic acid binding protein or sequence-specific endonuclease is detected using a single molecule detection system. Even more preferably, the single molecule detection system is a linear polymer analysis system, such as but not limited to a Gene Engine™ system, an optical mapping system, and a DNA combing system. The nucleic acid molecules may be analyzed in either a free form, e.g., in a flow system, or a fixed form.

In still another aspect, the invention provides a composition comprising a nucleic acid binding protein labeled to a single detectable label. As stated above, the nucleic binding protein may be a DNA binding protein, or an RNA binding protein. The nucleic acid binding protein may be selected from the group consisting of a sequence-specific endonuclease, a polymerase, a methylase, and a transcription factor.

In some embodiments, the nucleic acid binding protein is covalently labeled with the single detectable label. In others, the nucleic acid binding protein is ionically labeled with the single detectable label.

The detectable label may be selected from the group consisting of a fluorescent molecule, a chemiluminescent molecule, a radioisotope, an enzyme substrate, a biotin molecule, an avidin molecule, an electrical charged transducing molecule, a nuclear magnetic resonance molecule, a semiconductor nanocrystal, an electromagnetic molecule, an electrically conducting particle, a ligand, a chromogenic substrate, an affinity molecule, a quantum dot, a protein, a peptide, a nucleic acid, a carbohydrate, an antibody, an antibody fragment, an antigen, a hapten, and a lipid. In other embodiments, the detectable label is detected using a detection system such as those recited above.

In all aspects of the invention, the detectable label may be a non-bead label, or a non-fluorescently labeled bead.

All of the foregoing aspects and embodiments of the invention will be explained in greater detail herein.

These and other embodiments of the invention will be discussed in greater detail herein.

### BRIEF DESCRIPTION OF THE FIGURES

FIG. 1A is a representation of an autoradiogram showing the presence of complexes of BamHI/DNA complexes as a function of time in the presence of Ca²⁺ and in the absence of Mg²⁺.

FIG. 1B is a graph of the natural log of the ratio of bound nucleic acid to total nucleic acid as a function of time, and as used to calculate the half-life of the BamHI/DNA complex.

FIG. 2 is a representation of an autoradiogram showing the levels of NotI bound and unbound duplexes as a function of calcium concentration.

FIG. 3A is a schematic showing BamHII sites on a lambda phage DNA.

FIG. 3B is a graph showing the label average from lambda DNA bound by Alexa 546 BamHI.

FIG. 4A is a schematic showing EcoRI sites on a lambda phage DNA.

FIG. 4B is a graph showing the label average from lambda DNA bound by Alexa 546 EcoRI.

FIG. 5A is a schematic showing BamHI sites on a NotI large fragment ba12M9 (derived from a BAC).

FIG. 5B is a graph showing the label average from the NotI large fragment ba12M9 DNA bound by Alexa 546 BamHI.

It is to be understood that the Figures are not required to enable the invention.

### DETAILED DESCRIPTION OF THE INVENTION

The invention exploits the ability of certain proteins to bind a nucleic acid molecule without modifying it for labeling and sequencing purposes. Information can thereby be gained by analyzing for the presence or absence of a bound nucleic acid binding protein, or by determining the location and relative position of one or more bound proteins. These methods are not dependent upon the nucleic acid molecule being in a linear state. For example, the nucleic acid molecule can be analyzed in a compacted, non-linear state particularly when the only information to be gained is whether or not a protein is bound to a nucleic acid molecule.

Proteins suitable to these analyses bind to a nucleic acid molecule in a sequence-specific manner thereby allowing sequence information to be gained from such binding events. These proteins may be DNA or RNA binding proteins, or they may be capable of binding to both DNA and RNA. Examples of such proteins include but are not limited to polymerases such as DNA polymerase and RNA polymerase, methylases such as DNA methyltransferases, sequence-specific endonucleases such as EcoRI and BamHI, and sequence-specific transcription factors or repressors such as but not limited to GATA family members, Ikaros, NF-kappaB, SpI, Hox family members, MyoD, fos, jun, NFAT, nuclear hormone receptors, and the like.

The proteins are bound to nucleic acid under conditions that prevent their ability to modify the target nucleic acid. For example, an endonuclease is contacted to a nucleic acid molecule under non-cleaving conditions, as described below. A polymerase is contacted to a nucleic acid molecule in conditions that preclude synthesis of a new nucleic acid strand and more importantly translocation of the polymerase along the target nucleic acid molecule. Such conditions include but are not limited to an absence of unincorporated nucleotides or a primer. A methylase is preferably contacted to a nucleic acid molecule in conditions that preclude methylation of the target nucleic acid molecule. Such conditions may include but are not limited to the presence of methylase inhibitors such as 5-azacytidine (5-aza), 5-aza-2′deoxycytidine (also known as Decitabine in Europe), 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, 5-fluoro-2′deoxycytosine, sinefungin, and homocysteine.

The invention also provides compositions of nucleic acid binding proteins that are labeled with a single detectable label. These proteins are the same as those recited herein with respect to other aspects of the invention. A “single detectable label” refers to one detectable label rather than a plurality.

In particularly important aspects, the invention provides methods, compositions and systems for analyzing nucleic acids based on the use of sequence-specific endonucleases that are allowed to bind but not cut nucleic acid molecules under certain conditions. The pattern of binding of the endonucleases to a nucleic acid molecule can be used to derive sequence information.

The invention utilizes restriction endonucleases to tag their cognate recognition sequences. It has been found that when binding reactions are allowed to occur in the presence of calcium cations, little or no cleavage of the nucleic acid molecule takes place. Rather in the presence of calcium ions, the binding and stability of the restriction enzyme/DNA complex is enhanced. Other divalent cations can also be used and these include Co²⁺, Mn²⁺, etc. Thus, absence of Mg²⁺ cations alone appears insufficient for proper and efficient binding of sequence-specific endonucleases to a target nucleic acid molecule. Although not intending to be bound by any particular mechanism, it is possible that Ca²⁺ cations inhibit the cleavage reaction. Moreover, Ca²⁺ cations may also inhibit the dissociation of the endonuclease from the nucleic acid molecule.

Accordingly, the invention is based in some aspects on labeling strategies that label nucleic acids (preferably DNA molecules) under “non-cleaving conditions”. As used herein, the “non-cleaving condition” is a condition in which the sequence-specific endonuclease is able to bind to a nucleic acid molecule in a sequence-specific manner, but does not appreciably cleave the nucleic acid molecule. This means that less than 50%, less than 75%, less than 80%, less than 90%, less than 95%, less than 99%, or no nucleic acids are cleaved. This can be achieved by modulation of divalent cations (both type and concentration), temperature, pH, and the like. This also generally requires the presence of calcium cations. A non-cleaving condition may still contain a measurable amount of magnesium cations but this amount is insufficient for the restriction enzyme to appreciably cut the nucleic acid.

Non-cleaving conditions may vary depending upon the endonuclease, but generally they are characterized by having little or no Mg²⁺ present. They are also generally characterized as including another divalent cation such as but not limited to Ca²⁺, Co²⁺ or Mn²⁺. In preferred embodiments, the divalent cation is Ca²⁺. It may also be important that when Mg²⁺ and Ca²⁺ are both present, that the concentration of Mg²⁺ be orders of magnitude less than that of Ca²⁺. One reason for this is that endonucleases generally exhibit a greater affinity for Mg²⁺ than they do for Ca²⁺. Thus, the Ca²⁺ concentration may be at least or about 2, 3, 4, 5, 10, 20, 50, 100, 500, 1000, 2000, 5000 or 10000 fold greater than the Mg²⁺ concentration. Notwithstanding the foregoing however it is still preferred that the Mg²⁺ concentration be so low as to be insufficient to facilitate cleavage of the nucleic acid molecule. The Ca²⁺ concentration may be 5 nM although this amount can vary depending upon the concentration of Mg²⁺.

All restriction endonucleases require divalent cations for cleavage. The divalent cation that is almost exclusively required is magnesium, although manganese can be utilized by some restriction enzymes. The precise role of the divalent cation(s) in the catalysis of cleavage is controversial. The generalized mechanism is believed to involve the generation of a reactive nucleophile from a water molecule, the nucleophilic attack of the incipent hydroxide at the phosphate on the scissile phosphodiester bond, stabilization of the extra negative charge on the penta-coordinated intermediate, as well as the protonation of the leaving group (a 3′ OH in this case). The crystal structures of several of the restriction enzymes indicate the presence of a water molecule within the hydration sphere of the Mg²⁺. It is believed this water molecule may be involved in the protonation of the leaving group. The divalent metal may either aid in the generation of the reactive nucleophile or aid in the stabilization of a negative charge on the penta-coordinated intermediate. Sequence-specific binding in the presence of calcium has been observed with BamHI, BglI, BglII, EcoRI, EcoRV, MunI, PvuII, HaeIII and HinP1.

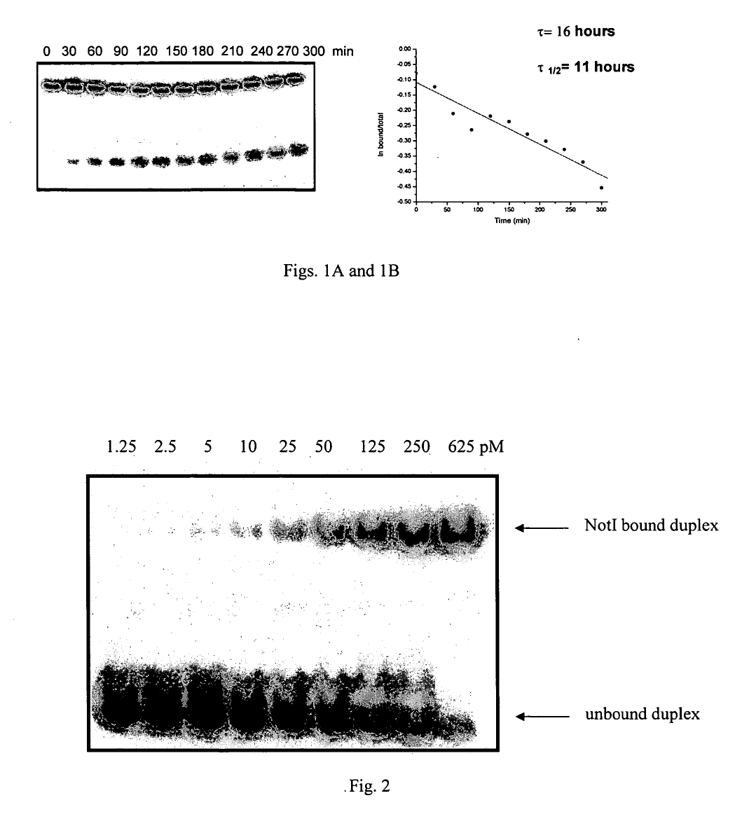

Kinetic analysis of the BamHI, EcoRI, and EcoRV complexes revealed that calcium not only enables specific binding of the restriction enzyme to its recognition site, but stabilizes the complex. The half-life of these complexes is on the order of several hours and no cleavage activity is observable. Data suggest that calcium has two effects on the kinetics of the endonuclease/DNA complex. First, low concentrations of calcium (0.1-0.2 mM) significantly increase the equilibrium association constant (Kₐ). This increase in Kₐ has been observed for EcoRI, EcoRV, BamHI and PvuII. Second, calcium strongly inhibits dissociation of the endonuclease from its recognition sequence. This increases the lifetime of enzyme/DNA complexes from a few seconds to several hours. The introduction of calcium into a binding reaction with EcoRV and a short DNA duplex caused a 740-fold increase in the complex lifetimes from 38 seconds to 28,000 seconds. The lifetime of BamHI binding to a short duplex in the presence of calcium is approximately 39,600 seconds (unpublished observations, see FIG. 1).

In order to locate the enzyme “tags” on a linear stretch of DNA, the restriction endonucleases must be visualized and assigned a position along the DNA backbone. The visualization of the restriction endonuclease will include covalent or non-covalent attachment of fluorescent or other detectable moieties (e.g., fluorophores, fluorospheres, quantum dots, GFP or fluorescent antibodies) to the endonuclease or nucleic acid binding protein. Positional information on the location of the fluorescent tags along the DNA can be achieved by using a labeled DNA molecule. The linearized DNA molecule may possess either end labels or a backbone stain. The position of the tags can be directly observed by analysis of the sample on a single molecule detector, such as the GeneEngine™

In still other embodiments, as discussed herein, the binding pattern can be oriented within a single nucleic acid molecule by staining the molecule for known sequences and structures such as telomeres, centromere, repetitive sequences such as Alu repeats, and the like. In still further embodiments, the nucleic acids may be further processed in order to label them for comparison with genomic maps. For example, the nucleic acid maps may be labeled with any label that has been previously used to map that particular genome. The nucleic acid so stained can then be compared to the genomic map generated using the same label. As a specific example, the nucleic acid may be labeled with probes that bind to repetitive sequences, such as Alu repeats, and then compared with an Alu map of the entire genome in order to determine the location and orientation of the nucleic acid molecule. Once this is determined, the location of binding of the nucleic acid binding protein or sequence-specific endonuclease can be determined with respect to the genomic map.

The genomic maps can be obtained for public databases including the Human Genome Project, the results of which are available from the NCBI or NIH websites. These genomic maps can be sequence maps at various levels of resolution, or they can be motif maps, or structural maps, but they are not so limited.

The term “nucleic acid” is used herein to mean multiple nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)), or an inosine (I). As used herein, the term refers to oligoribonucleotides as well as oligodeoxyribonucleotides. The term shall also include polynucleosides (i.e., a polynucleotide minus a phosphate) and any other organic base containing polymer. Nucleic acid molecules can be obtained from existing nucleic acid sources (e.g., genomic DNA or RNA), or by synthetic means (e.g. produced by nucleic acid synthesis, recombinant DNA techniques, or amplification reactions).

More specifically, DNA is a double stranded polymer comprised of phosphodiester linked pentose deoxyribose sugars with attached purine or pyrimidine nitrogenous bases. The asymmetry in the pentose sugar leads to a directionality in the phosphodiester linkage whereby the sugar units are linked by phosphodiester bonds between 5′ and 3′ carbons of different sugar units. Thus the two polymer strands of a DNA molecule are anti-parallel. There are two purines (adenine and guanine) and two pyrimidines (thymine and cytosine) that constitute the types of nitrogenous bases that are naturally attached to the sugars. Adenine preferentially hydrogen bonds to thymine and cytosine preferentially hydrogen bonds to guanine. The sequence of a nucleic acid molecule refers to the order of the bases along its length. The order of the bases on any one strand determines (or alternatively, is determined by) the order of bases on the other strand by the anti-parallel nature of the DNA strands and by the complementary nature of the hydrogen bonding that can occur between the appropriate purine and pyrimidine bases.

A “nucleic acid molecule” can be a DNA, or an RNA, whether single or double stranded. The terms “nucleic acid” and “nucleic acid molecule” are used interchangeably. DNA includes genomic DNA (such as nuclear DNA and mitochondrial DNA), as well as in some instances cDNA. The RNA can be mRNA, rRNA, snRNA, RNAi, miRNA, siRNA, and the like. It is to be understood that the reference to DNA in the exemplifications described herein are merely for convenience and clarity, and that any nucleic acid molecule, including those recited above, can be processed and analyzed as described herein. The nucleic acid molecule can be any size, including several nucleotides in length, several hundred, several thousand, and even several million nucleotides in length. In some embodiments, the nucleic acid molecule is the length of a chromosome.

The methods of the invention may be performed in the absence of prior nucleic acid amplification in vitro. In some preferred embodiments, the nucleic acid molecule is directly harvested and isolated from a biological sample (such as a tissue or a cell culture) without amplification of the nucleic acid molecule. Accordingly, some embodiments of the invention involve analysis of “non in vitro amplified nucleic acid molecules”. As used herein, a “non in vitro amplified nucleic acid molecule” refers to a nucleic acid molecule that has not been amplified in vitro using techniques such as polymerase chain reaction or recombinant DNA methods.

A non in vitro amplified nucleic acid molecule may, however, be a nucleic acid molecule that is amplified in vivo (e.g., in the biological sample from which it was harvested) as a natural consequence of the development of the cells in the biological sample. This means that the non in vitro nucleic acid molecule may be one which is amplified in vivo as part of gene amplification, which is commonly observed in some cell types as a result of mutation or cancer development.

Harvest and isolation of nucleic acids are routinely performed in the art and suitable methods can be found in standard molecular biology textbooks. The nucleic acid molecule may be harvested from a biological sample such as a tissue or a biological fluid. The term “tissue” as used herein refers to both localized and disseminated cell populations including. but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

In some embodiments, the invention can be used to analyze nucleic acid derivatives. As used herein, a “nucleic acid derivative” is a non-naturally occurring nucleic acid molecule. Nucleic acid derivatives may contain non-naturally occurring elements such as non-naturally occurring nucleotides and non-naturally occurring backbone linkages.

Nucleic acid derivatives may include substituted purines and pyrimidines such as C-5 propyne modified bases (Wagner et al., Nature Biotechnology 14:840-844, 1996). Purines and pyrimidines include but are not limited to adenine, cytosine, guanine, thymidine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Nucleic acid derivatives also include peptide nucleic acids (PNAs) and locked nucleic acids (LNAS). Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, nucleic acid derivatives may include a 2′-O-alkylated ribose group. In addition, modified nucleic acids may include sugars such as arabinose instead of ribose. Thus the nucleic acid derivatives may be heterogeneous in backbone composition thereby containing any possible combination of polymer units linked together. In some embodiments, the nucleic acids are homogeneous in backbone composition.

Non-naturally occurring backbone linkages include but are not limited to phosphorothioate linkages, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

As stated above, the methods of the invention can be performed on nucleic acid molecules in various states. For example, the nucleic acid molecule may be in flow or it may be fixed to a solid surface. As another example, the nucleic acid molecule may be linearized prior to or during the analysis but the methods are not so limited. Rather it is possible to derive valuable information from non-linearized nucleic acid molecules as well. This is referred to as direct molecular analysis (as compared to direct linear analysis which requires linear nucleic acid molecules). Direct molecular analysis can be used to determine whether a nucleic acid molecule contains a particular sequence such as a polymorphism or a mutation. The presence or absence of this sequence can be determined based on the presence or absence of binding of the nucleic acid binding protein that binds specifically to that sequence. These analyses are not particularly concerned with the location or relative positioning of such sequences. However, they may provide valuable information on the number of such sequences that are present in the nucleic acid molecule. This latter embodiment can be achieved by measuring the level of signal from the nucleic acid molecule as the signal level will correlate with the number of nucleic acid binding proteins bound to the molecule.

The method is not limited to analysis of a single nucleic acid binding protein. Rather, a number of nucleic acid binding proteins may be contacted with a nucleic acid binding protein at a given time and in some instances each type of protein is distinctly labeled from the others.

Some aspects of the invention use single molecule detection systems. Other aspects of the invention use linear polymer analysis systems in order to detect the nucleic acid molecules and the pattern of binding of the nucleic acid binding protein or the sequence-specific endonuclease to the nucleic acid molecule. A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed as a histogram (signal intensity vs. time) that can then be translated into a map with knowledge of the velocity of the nucleic acid molecule. It is to be understood that in some embodiments, the nucleic acid molecule is attached to a solid support, while in others it is free flowing. In either case, the velocity of the nucleic acid molecule as it moves past, for example, an interaction station or a detector, will aid in determining the position of the labels, relative to each other and relative to other detectable markers that may be present on the nucleic acid molecule.

Accordingly, the linear polymer systems are able to deduce not only the total amount of label on a nucleic acid molecule, but more importantly for some embodiments, the location of such labels. The ability to locate and position the labels allows the binding patterns to be superimposed on other genetic maps, in order to facilitate sequencing.

Other single molecule nucleic acid analytical methods which involve elongation of DNA molecule can also be used in the methods of the invention. These include optical mapping (Schwartz et al., 1993, Science 262:110-113; Meng et al., 1995, Nature Genet. 9:432; Jing et al., Proc. Natl. Acad. Sci. USA 95:8046-8051) and fiber-fluorescence in situ hybridization (fiber-FISH) (Bensimon et al., Science 265:2096; Michalet et al., 1997, Science 277:1518). In optical mapping, nucleic acid molecules are elongated in a fluid sample and fixed in the elongated conformation in a gel or on a surface. Restriction digestions are then performed on the elongated and fixed nucleic acid molecules. Ordered restriction maps are then generated by determining the size of the restriction fragments. In fiber-FISH, nucleic acid molecules are elongated and fixed on a surface by molecular combing. Hybridization with fluorescently labeled probe sequences allows determination of sequence landmarks on the nucleic acid molecules. Both methods require fixation of elongated molecules so that molecular lengths and/or distances between markers can be measured. Pulse field gel electrophoresis can also be used to analyze the labeled nucleic acid molecules. Pulse field gel electrophoresis is described by Schwartz et al. in Cell, 1984, 37:67. Other nucleic acid detection systems are described by Otobe et al. (NAR, 2001, 29:109), Bensimon et al. in U.S. Pat. No. 6,248,537, issued Jun. 19, 2001, Herrick and Bensimon (Chromosome Res 1999, 7(6):409-423), Schwartz in U.S. Pat. No. 6,150,089 issued Nov. 21, 2000 and U.S. Pat. No. 6,294,136, issued Sep. 25, 2001.

In some aspects, a Gene Engine™ system is used to interrogate nucleic acid molecules. Gene Engine™ technology is described in greater detail in published PCT patent applications having serial numbers WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other patents, applications and references recited herein are incorporated by reference in their entirety. This system is capable of determining the spatial location of sequence-specific tags along a nucleic acid molecule. A map of specific sequences within the nucleic acid molecule can be derived from the relative spatial location of the tags. The spatial location is determined by interrogating nucleic acid molecules, preferably single molecules, with a detection system that corresponds to the labels on the sequence-specific tag. The sensitivity of the afore-mentioned system allows single nucleic acids to be studied.

A “sequence-specific endonuclease” is an enzyme that cleaves nucleic acids in a sequence-specific manner under suitable conditions. “Sequence-specific” as used herein means that the enzyme recognizes a particular linear arrangement of nucleotides or derivatives thereof, and cleaves the backbone (e.g., creates a single or double stranded cut) within that arrangement or in the vicinity of that arrangement. In some important embodiments, the cut is double stranded. Commonly, the sequence-specific restriction enzyme cleaves the backbone within the same sequence it recognizes.

Many Type II restriction enzymes are dimers of two identical subunits that form one DNA binding site and two catalytic units that cleave symmetrically within the recognition sequence. The catalytic site is active only when the restriction enzyme is bound to its double stranded DNA substrate at its specific recognition site. EcoRV has been a prototypical restriction enzyme for study and has been the subject of considerable mechanistic and structural studies and protein engineering. (Stahl F., W. Wende, A. Jeltsch and A. Pingoud PNA 93:6175-6180 1996.)

The method can also be carried out using homing endonucleases. Homing endonucleases are enzymes that recognize specific DNA sequence sites of 35 base pairs or more and catalyze a double DNA strand break within the recognition site under the appropriate conditions. These enzymes are involved in insertion and excision of genetic elements. The family of homing endonucleases can be divided into 4 sub-families characterized by the following motifs: (1) LAGLIDADG, (2) GIY-YIG, (3) H—N—H and (4) His-Cys. Homing endonucleases may be either dimeric or monomeric. One such typical monomeric homing endonuclease, PI-SceI has two copies of an LAGLIDADG motif in what appears to be two distinct catalytic subunits. Each subunit was shown to specifically catalyze the cleavage of the top and bottom strand respectively of the double stranded substrate. The monomeric homing endonuclease PI-SceI has two catalytic centers for cleavage of the two strands of its DNA substrate. (EMBO 18:6908-6916, 1999.)

It is clear that several if not all sequence-specific endonucleases can be used in the methods of the invention, under conditions in which the endonuclease binds in a sequence-specific manner to the nucleic acid molecule but does not appreciably cleave it.

Sequence-specific endonucleases also include synthetic restriction endonucleases that have been engineered by combining a DNA binding motif of one protein with a cleaving motif of another. DNA binding protein motifs such as zinc finger motifs, homeobox binding domains, lac repressor, GAL, cro etc. can be fused with DNA cleavage domains to construct sequence-specific restriction enzymes. Such chimeric restriction enzymes have been built and described. Yang-Gyun K. and Chandrasegaran S. (PNAS 91:883-887, 1994) reported fusing the Drosophila Ultrabithorax homeodomain to the cleavage domain of Fok I restriction enzyme. More relevantly, chimeric restriction enzymes can be built from fusing zinc finger domains to the cleavage domain of Fok I and thereby building sequence-specific restriction enzymes.

Transposases can also be used to label nucleic acids at discrete sequence sites. Transposases are enzymes involved in moving transposons around in a genome. The sequence-specific DNA binding characteristics of the transposases can be exploited according to the invention.

The nucleic acid binding proteins (including sequence-specific endonucleases) are detectable. They may be inherently or intrinsically detectable (e.g., auto fluorescing) or extrinsically manipulated to be detectable. Thus in some embodiments, the nucleic acid binding proteins, sequence-specific endonucleases and/or the nucleic acid molecule are labeled with a detectable label. The proteins or endonucleases may be covalently or ionically labeled with the detectable label. Generally, detection of a label involves absorbance or emission of energy by the label. The label can be detected directly by its ability to emit and/or absorb light of a particular wavelength. An example of direct detection is the use of a fluorophore that absorbs light of a particular wavelength, and emits light of commonly a longer wavelength. Alternatively, the label can be detected indirectly by its ability to bind, recruit and, in some cases, cleave another moiety which itself may emit or absorb light of a particular wavelength. An example of indirect detection is the use of a first enzyme label which cleaves a substrate into visible products.

The label may be of a chemical, peptide or nucleic acid nature although it is not so limited. Other examples of labels include but are not limited to radioactive isotopes such as P³² or H³, chemiluminescent substrates, chromogenic substrates, fluorescent markers such as fluorochromres (e.g., fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), etc.), optical or electron density markers, biotin, avidin, digoxigenin, epitope tags such as the FLAG epitope or the HA epitope, and enzyme tags such as alkaline phosphatase, horseradish peroxidase, β-galactosidase, etc. Also envisioned by the invention is the use of semiconductor nanocrystals such as quantum dots, described in U.S. Pat. No. 6,207,392 as labels. Quantum dots are commercially available from Quantum Dot Corporation as well as others.

In some instances, the detectable label is not a bead or a particle capable of being loaded with a plurality of detectable labels. For example, the detectable label may be a bead or particle provided the bead or particle is itself inherently detectable and has not been loaded a priori with a plurality of detectable labels such as fluorescent moieties. In other embodiments, the detectable label is a non-fluorescently labeled bead or particle. A magnetic particle is an example of a non-fluorescently labeled bead or particle.

The labels may be directly or indirectly linked to the protein, endonuclease or nucleic acid molecule.

Analysis of the nucleic acid molecule involves detecting signals from the labels, and in some instances determining the position of those labels. In some instances, it may be desirable to further label the nucleic acid molecule with a standard marker that facilitates comparing the information so obtained with that from other nucleic acids analyzed or with genomic maps. For example, the standard marker may be a backbone label, a label that binds to a particular sequence of nucleotides (whether unique or not), or a label that binds to a particular location in the nucleic acid molecule (e.g., an origin of replication, a transcriptional promoter, a centromere, etc.).

One subset of backbone labels are nucleic acid stains that bind nucleic acids in a substantially sequence-independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS75 1, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

The linear polymer analysis system is equipped with a detection system that is chosen to correspond to the type of labels used. The labels emit signals that are detected in a spatial or temporal manner. As an example of one suitable system, the Gene Engine™ system allows single nucleic acid molecules to be passed through an interaction station in a linear manner. The nucleotides are interrogated individually in order to determine whether they are conjugated to a detectable label. Interrogation involves exposing the nucleic acid molecule to an energy source such as optical radiation of a set wavelength. In response to the energy source exposure, the detectable label on the nucleotide emits a characteristic detectable signal. The linear polymer analysis system can also be an optical mapping system, such as a DNA combining system.

The mechanism for signal emission will depend on the type of label. The detection system can be selected from the group of detection systems consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system, but is not so limited.

The invention embraces the use of any combination of labels along the length of a nucleic acid molecule. This means that a nucleic acid molecule may be labeled with, for example, a fluorophore, a chromophore, a nuclear magnetic resonance label and a semiconductor nanocrystal along its length and it may be analyzed by the systems described herein. The linear polymer analysis systems have the capability of detecting signals from a number of different “signal modalities”. In one important embodiment, the system uses laser induced fluorescent detection to determine the location of a sequence defined by fluorescent labels.

The sequence-specific information may be either on a single molecule or on a population of molecules. Thus, it is not necessary to label all of the sequence-specific sites on a molecule. If there is a homogenous population of molecules then it is possible to partially label members of the population and then reassemble the data to generate a complete map for a particular sequence provided that the individual members can be aligned. This method effectively creates a population of single DNA molecule data with a “nested” set of sequence-specific data.

Each nucleic acid molecule so labeled will have a unique pattern of binding by the endonuclease. This unique pattern can be akin to a “fingerprint” of the nucleic acid molecule. The greater the number of different endonucleases used (each with a distinct recognition sequence), the more sequence information is available.

The sequencing information derived using the methods of the invention can be compared to genomic sequencing information that is available from sources such as the human genome project. The binding patterns deduced using the methods of the invention can also be superimposed onto physical genomic maps. These maps (including sequence, motif and structural maps) are available from public sources such as the human genome project, or the genome sequencing projects of other organisms. Superimposition of either or both the sequencing information or the restriction enzyme binding patterns helps to orient such information and thus identify the region of the genome that is being analyzed. The physical maps of genomes are therefore used as references for orienting the binding patterns determined using the methods of the invention. Moreover, it also helps to identify the genetic loci that are bound. All aspects of the invention can include the step of comparing the restriction enzyme binding pattern to a physical map of the genome or part thereof for that particular species.

### References

Pingoud, A., and A. Jeltsch Structure and function of type II restriction endonucleases. Nucleic Acids Research 29, 3705-3727 (2001).
Sam, M. D., and J. J. Perona Catalytic roles of divalent metal ions in phosphoryl transfer by EcoRV endonuclease. Biochemistry 38, 6576-6586 (1999).
Fox, K. R. DNAase I footprinting of restriction enzymes. Biochem. Biophys. Res. Commun. 155, 779-785 (1988).
Vipond, I. B., and S. E. Halford Specific DNA recognition by EcoRV endonuclease induced by calcium ions. Biochemistry 34, 1113-1119 (1995).
Vipond, I. B., Baldwin, G. S., and S. E. Halford Divalent metal ions at the active sites of the EcoRV and EcoRI restriction endonucleases. Biochemistry 34 (1995).
Engler, L. E., Welch, K. K., and L. Jen-Jacobson Specific binding by EcoRV endonuclease to its DNA recognition site GATATC. Journal of Molecular Biology 269, 82-101 (1997).
Conlan, L. H., and C. M. Dupureur Dissecting the metal ion dependence of DNA binding by PvuII endonuclease. Biochemistry 41, 1335-1342 (2002).
Lesser D. R., K., M. R., and L. Jen-Jacobson. The energetic basis of specificity in the EcoRI endonuclease-DNA interaction. Science 250, 776-786 (1990).
Soslau G. and Pirollo P. 1983 Selective inhibition of restriction endonuclease cleavage by DNA intercalators Biochem Biophys Res Commun 1983 115(2): 484-491
Osterlund M, Luthman H. Nilsson S V, Magnusson G. Ethidium-bromide inhibited restriction endonucleases cleave one strand of circular DNA Gene 1982 20(1):121-125
Nakatani K., Sando S., Saito I. Photoinduced cleavage of single and double stranded DNA at single guanine proximal to target sequence by dibenzoyldiazomethane-ODN conjugate Nucleic Acids Symp Ser 37, 85-6, 1997
Pan C Q, Landgraf R., Sigman D S DNA-binding proteins as site specific nucleases Mol Microbiol 1994 12(3):335-42
Kittler L., Bell A., Baguley B C., Lober G. Sequence-specific modulation of DNA restriction enzyme cleavage by minor groove binders Biol Chem 379: 4-5 1998
Fukuda H., Ohtsuba E, Roles of TraI protein with activities of cleaving and rejoining the single-stranded DNA in both initiation and termination of conjugal DNA transfer Genes Cells 2:735-751 1997
Cost G J. Golding A, Schlissel M S, Boeke J D Target DNA chromatinization modulates nicking by L1 endonuclease NAR 29:573-577 2001 L1 endonuclease (L1 EN) nicks genomic DNA as step in transposition reaction
Champoux J J DNA topoisomerase I mediated nicking of circular duplex DNA Methods Mol Biol 2001 95:81-87

### EXAMPLES

### Example 1

100 pM of a radiolabeled DNA duplex containing a single BamHI recognition sequence was incubated with 2 nM BamHI in the presence of 5 mM CaCl₂. At the indicated time points a 6000-fold molar excess of unlabeled specific competitor DNA was added. Least squares analysis of complex dissociation data suggests the half-life of the complex is approximately 39,600 minutes.

### Example 2

The equilibrium binding of the NotI, PmeI and EagI restriction endonucleases in the presence of calcium was investigated. Preliminary data indicate these endonucleases bind to their target recognition site tightly in the presence of calcium with no appreciable cut-through activity observed (see FIG. 2). Thus the inclusion of calcium in a restriction endonuclease reaction may be a general method to stimulate tight complex formation while inhibiting cleavage.

### Example 3

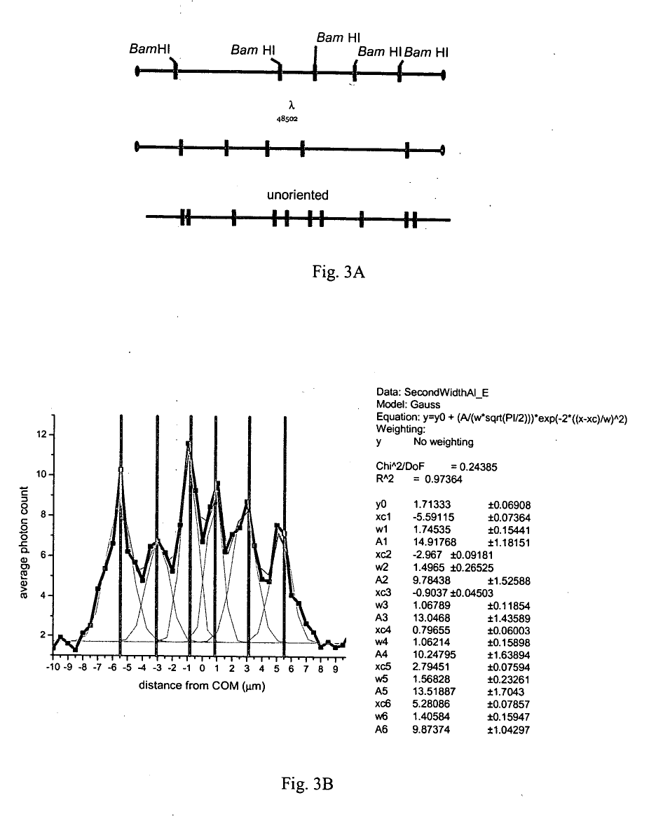

As shown in FIG. 3A lambda DNA has five BamHI binding sites. The positions of the tag locations expected on a population of stretched but unoriented lambda DNA molecules is shown on the bottom molecule. The blue oval represents the “head” of the lambda DNA molecule, while the purple oval represents the tail. Since molecules are not oriented prior to analysis, they are able to enter the detection spot in either a “head-first” or “tail-first” orientation.

FIG. 3B shows the label average from lambda DNA bound by Alexa 546 BamHI. The label average sums the photon counts along the DNA backbone of those DNA molecules selected for analysis. The peaks represent positions, in micrometers from the center of mass (COM), along the lambda DNA backbone where increased photon emission is observed. These molecules are unoriented prior to analysis and thus expected to enter the detection spot in both “head-first” and “tail-first” orientations. Expected BamHI tag locations, as indicated by the lambda DNA sequence, are indicated by red lines. The data is fit to Gaussians and the means of the normal curves are used to indicate the observed tag location. The observed tag positions are in good agreement with the expected and the average difference is less than 0.2 micrometers.

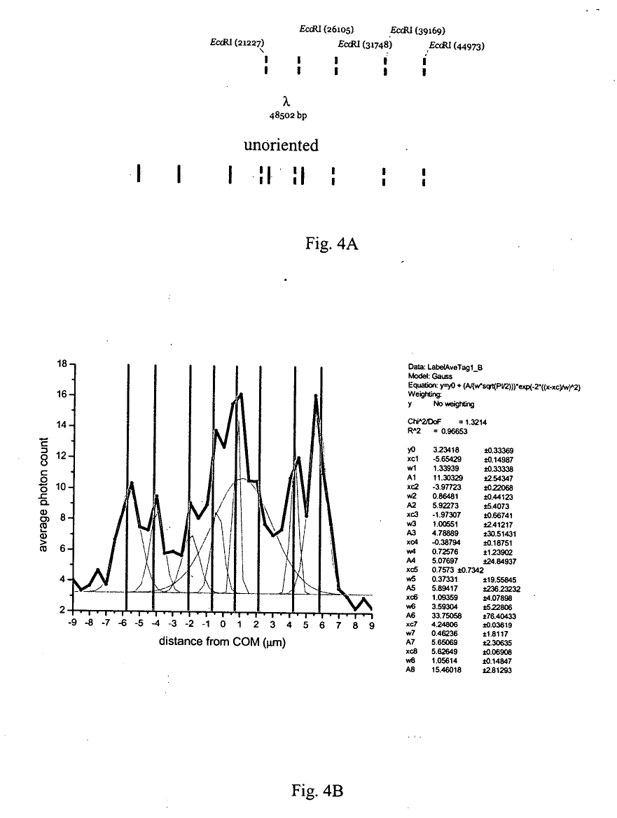

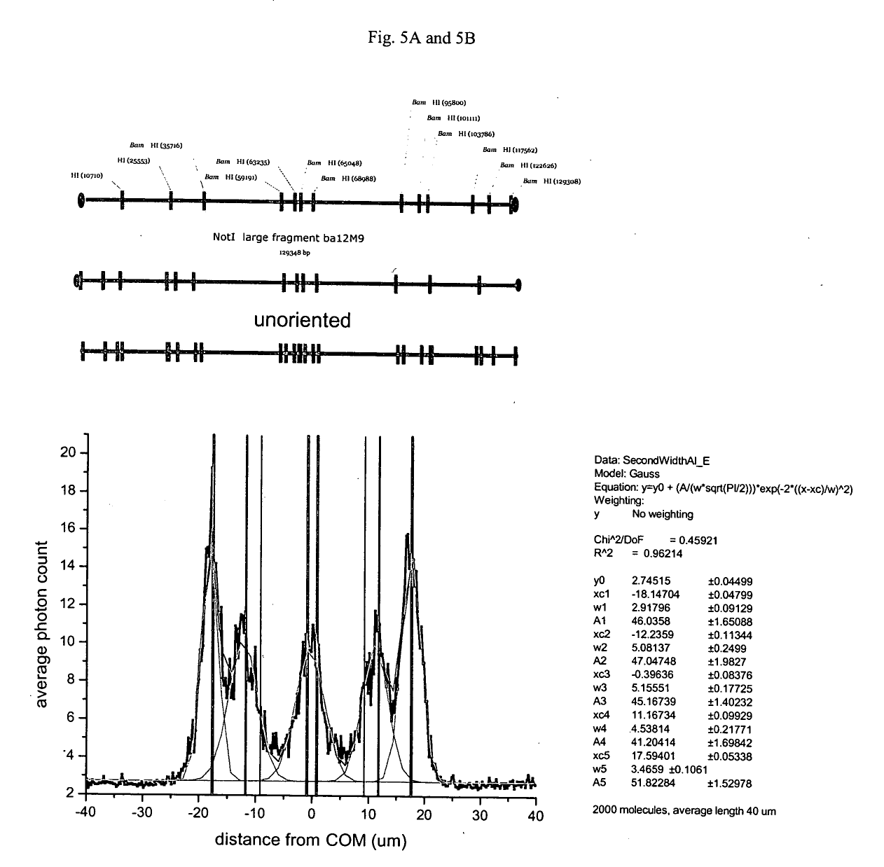

A similar analysis was performed on lambda DNA with respect to EcoRI binding sites. FIG. 4A illustrates that lambda DNA has five EcoRI binding sites. The expected locations of EcoRI binding on an unoriented DNA molecule are indicated. FIG. 4B shows the label average from lambda DNA bound by Alexa 546 EcoRI. The expected tag positions are indicated by the red lines. Observed tag positions are in good agreement with the expected tag positions with the average difference being less than 0.5 micrometers. Note the difference in barcode patterns between the BamHI bound lambda DNA molecules and the EcoRI bound lambda DNA molecules.

A similar analysis was performed on the bacterial artificial chromosome (BAC) RP11 12M9 digestion fragment. FIG. SA illustrates that the NotI large fragment ba12M9 (129348 bp) DNA has thirteen BamHI binding sites. Blue ovals indicate the “head” of the DNA molecule while the purple oval indicates the “tail”. Tag locations on a population of unoriented molecules are indicated. FIG. 5B shows the label average from the NotI large fragment ba12M9 DNA bound by Alexa 546 BamHI. The label average contains peaks that closely correspond to the expected tag locations with the average distance between an observed peak and an expected peak of less than 0.5 micrometers.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

1. A method for analyzing a nucleic acid molecule, comprising:
contacting a nucleic acid molecule with a detectable sequence-specific endonuclease in a non-cleaving condition for a time sufficient for the sequence-specific endonuclease to bind to the nucleic acid molecule in a sequence-specific manner, and detecting the bound sequence-specific endonuclease.

2. The method of claim 1, wherein the sequence-specific endonuclease is a restriction endonuclease.

3. The method of claim 2, wherein the restriction endonuclease is a type II restriction endonuclease.

4. The method of claim 3, wherein the type II restriction endonuclease is selected from the group comprising BamHI, BglI, BglII, EcoRI, EcoRV, MunI, PvuII, HaeIII, HinPI, NotI, PmeI and EagI.

5. The method of claim 1, wherein the non-cleaving condition comprises a Mg²⁺ concentration that does not allow cleavage of the nucleic acid molecule.

6. The method of claim 5, wherein the Mg²⁺ concentration is zero.

7. The method of claim 1, wherein the non-cleaving condition comprises the presence of a divalent cation selected from the group consisting of Ca²⁺, Co²⁺ and Mn²⁺.

8. The method of claim 1, wherein the non-cleaving condition comprises the presence of Ca²⁺.

9. The method of claim 1, wherein the non-cleaving condition comprises a Ca²⁺ concentration that exceeds a Mg²⁺ concentration.

10. The method of claim 9, wherein the Ca²⁺ concentration exceeds the Mg²⁺ concentration by at least 50-fold, at least 100-fold, at least 500-fold, at least 1000-fold, at least 2000-fold or at least 5000-fold.

11. The method of claim 9, wherein the Ca²⁺ concentration exceeds the Mg²⁺ concentration by at least 500-fold.

12. The method of claim 8, wherein the Ca²⁺ concentration is about 5 nM.

13. The method of claim 1, wherein the nucleic acid molecule is a DNA or an RNA.

14. The method of claim 13, wherein the DNA is genomic DNA selected from the group consisting of nuclear DNA and mitochondrial DNA.

15. The method of claim 13, wherein the DNA is cDNA.

16. The method of claim 13, wherein the RNA is mRNA, rRNA, snRNA, RNAi, miRNA or siRNA.

17. (canceled)

18. The method of claim 1, wherein the sequence-specific endonuclease is labeled with a detectable label.

19-25. (canceled)

26. The method of claim 1, wherein the bound sequence-specific endonuclease is detected using a single molecule detection system that is a linear polymer analysis system.

27-32. (canceled)

33. A method for analyzing a nucleic acid molecule, comprising:
contacting a nucleic acid molecule with a detectable nucleic acid binding protein in a binding condition for a time sufficient for the detectable nucleic acid binding protein to bind to the nucleic acid molecule in a sequence-specific manner, and
detecting the bound detectable nucleic acid binding protein.

34-58. (canceled)

59. A composition comprising a nucleic acid binding protein labeled with a single detectable label.

60-67. (canceled)
